(19) United States
(12) Patent Application Publication
(10) Pub. No.: US 2007/0021808 A1
(43) Pub. Date: Jan. 25, 2007

(54) Patient cooling system and method

(76) Inventor: Arturo R. Rojas, Miami, FL (US)

Correspondence Address: HOLLAND & KNIGHT LLP, 10 ST. JAMES AVENUE, BOSTON, MA, 02116, US

(21) Appl. No.: 11/527,968

(22) Filed: Sep. 26, 2006

Related U.S. Application Data
(63) Continuation of application No. 11/081,409, filed on Mar. 16, 2005.
(60) Provisional application No. 60/553,388, filed on Mar. 16, 2004.

(51) Int. Cl. A61F 7/00; A61F 7/12

(52) U.S. Cl. 607/105; 607/113

(57) ABSTRACT

A system and method of reducing or raising patient body temperature in an expedited yet safe, cost-effective, and convenient manner. The patient cooling system of the present invention includes a positive pressure device, a cooler for regulating the temperature of gas entering a patient's lungs, and temperature monitoring and controlling means.

### CROSS-REFERENCE TO RELATED APPLICATIONS

This application is a continuation of and claims the benefit under 35 U.S.C. § 120 of U.S. application Ser. No. 11/081,409 filed Mar. 16, 2005, which claims the benefit under 35 U.S.C. 119(e) of U.S. Provisional Application Ser. No. 60/553,388 filed Mar. 16, 2004, the entire contents of which are hereby incorporated by reference.

### FIELD OF INVENTION

The present invention relates generally to the field of therapeutic hypothermia, and more particularly, to a system and method of reducing or increasing patient body temperature.

### BACKGROUND

Under normal circumstances, the human body maintains a near constant temperature of about 37 degrees Celsius or 98.6 degrees Fahrenheit maintaining a delicate balance that optimizes cellular functions and biochemical reactions and balances the heat lost to the environment by heat produced within the body.

There are a number of instances, however, where medical intervention is required to manipulate the core human body temperature of a patient. Particularly, there are circumstances under which a patient will need to be cooled in a rapid manner to thwart the onset of serious, and often fatal, repercussions. For instance, a patient may be suffering from malignant hyperthermia, a life threatening elevation in body temperature experienced by some patients after receipt of certain muscle relaxants and general anesthetics during surgery. This situation is called a pharnacogenetic reaction; a variation in drug response caused by hereditary factors. Such a rapidly progressive reaction is often fatal, and requires immediately initiated treatment. This condition can be reversed if the patient's core body temperature is immediately lowered to within acceptable parameters.

By slowing down a patient's metabolism, the demand for oxygen and nutrients can be minimized until appropriate treatment is effectuated. A dangerously high core body temperature is often due to infection, tumor necrosis, or malignant hyperthermia.

Such conditions result in harmful fluid and electrolyte imbalances, increased cellular metabolic rates, and cognitive impairment. If not immediately addressed, a patient may suffer irreversible cellular injury, loss of brain and liver cells, and ultimately may suffer critical organ failure resulting in death.

Evidence suggests that patient cooling provides beneficial protection against further deterioration of patient health in instances of cardiac arrest, surgery on the brain blood vessels, stroke, traumatic injury, or open heart operations. Cooling the blood before or during such events has been found to substantially decrease the severity of the resulting injury to the patient.

Recently, the American Heart Association recommended that some victims of heart attacks be chilled. There are about 250,000 to 300,000 people suffering from cardiac arrest in the United States yearly, with about 50,000 to 75,000 making it to the hospital with adequate time for blood cooling to protect the brain and heart from further injury.

Although the benefits of patient cooling are well known, existing methods and systems are cumbersome, ineffective, and completely inadequate for rapid patient cooling.

Current methods of cooling treatment include crude improvised solutions such as packing a patient in ice, or immersing the patient in cool water. Naturally, it is seen that such techniques, although well-intentioned, do not provide for rapid body temperature cooling as often required in surgery and Intensive care situations. Such treatment is difficult and labor intensive and cannot be performed in cases where time is of the essence.

Other attempts at patient cooling have included convective thermal blankets, room coolers, and other similar external cooling mechanisms. Although such devices do assist in cooling the environment surrounding a patient, they are generally ineffective in adequately reducing a patient's core body temperature. Furthermore, such methods generally produce unwanted patient shivering and discomfort which may even lead to an increase in core body temperature.

Evaporative cooling has also been attempted by wetting a patient's skin or clothing and allowing the water, or other liquid, to evaporate and remove heat from the body. Such treatment generally includes sponge baths and is sometimes combined with enhanced room air circulation to increase the rate of evaporation. Such cooling is not practical in intensive care situations, is extremely time-consuming and labor intensive, and inadequate for serious life-threatening conditions.

A variety of surgical patient blood cooling methods and systems are also available. Such treatment generally involves catheters inserted into a vein for direct cooling of a patient's blood. Such devices are invasive and require surgical incision. The invasive surgical treatment required by such devices require substantial time and skill to administer properly, force patients to undergo additional pain and discomfort, introduce the risk of contamination and blood clotting, and have been cost-prohibitive and impractical in use.

In these respects, the patient cooling system for medical treatment of the present invention substantially departs from the conventional concepts and designs of the prior art, and in so doing, provides a system and method capable of reducing patient body temperature in an extremely expedited yet substantially safe, cost-effective, and practical manner.

### SUMMARY

According to an embodiment of the invention, a system and method of regulating patient body temperature in an expedited yet substantially safe, cost-effective, and convenient manner uses a temperature regulated gas delivered to the lungs of a patient.

In general, in one embodiment, the invention features a temperature regulating system that includes a gas delivery device configured to deliver gas to a patient. A temperature regulating device is in fluid communication with the gas delivery device to regulate the temperature of the gas. A temperature sensor monitors the temperature of the gas, and a temperature controller enables control of the temperature of the gas.

In other embodiments, the gas delivery device is a positive pressure device, such as a ventilator, an anesthesia machine, an ambu bag, a continuous positive airway pressure machine, or a bi-level positive airway pressure machine.

In some embodiments, the temperature regulating device is a cooling device. In other embodiments, the cooling device may be an ice bag, a refrigerant based device, a thermo-electric cooler, an air-to-water cooler, a carbon dioxide cooler, or any combination thereof. In other embodiments, the temperature regulating device is a heating device. In still other embodiments, the temperature regulating device includes a heating device.

In further embodiments, the temperature sensor may be a thermometer or a thermocouple. In embodiments, the temperature controller is a temperature control dial connected to the temperature regulating device. In other embodiments, the temperature controller is in communication with the temperature sensor and the temperature regulating device to regulate the temperature of the gas to a preset temperature. In still other embodiments, the temperature regulating system also includes a second temperature sensor configured to monitor the temperature of the gas leaving the patient.

In further embodiments, the temperature regulating system also includes an inspiratory limb and an expiratory limb in communication with the gas delivery device. In embodiments, the temperature regulating device is in fluid communication with the inspiratory limb.

In general, in another embodiment, the invention features a temperature regulating system that includes a positive pressure device configured to deliver gas to a patient with an inspiratory limb and an expiratory limb in fluid communication with the positive pressure device. A temperature regulating device is in fluid communication with the inspiratory limb and configured to regulate the temperature of the gas. A temperature sensor is in fluid communication with the inspiratory limb and configured to monitor the temperature of the gas. A temperature controller is in communication with the temperature regulating device to control the temperature of the gas.

In further embodiments, the positive pressure device may be a ventilator or an anesthesia machine.

In further embodiments, the temperature regulating device is a cooling device, such as an ice bag, a refrigerant based device, a thermo-electric cooler, an air-to-water cooler, a carbon dioxide cooler, or any combination thereof. In other embodiments, the temperature regulating device is a heating device. In other embodiments, the temperature regulating device further includes a heating device.

In embodiments, the temperature regulating system also includes an inspiratory filter in fluid communication with the inspiratory limb, an expiratory filter in fluid communication with the expiratory limb, and a collector vial disposed within the expiratory limb that is configured to remove humidity and condensation from the temperature regulating system.

In general, in another embodiment, the invention features a method for treating a patient's body temperature that includes providing a positive pressure device to deliver a flow of gas to a patient's lungs and regulating the temperature of the gas flowing to the patient's lungs to regulate the patient's body temperature.

In further embodiments, the positive pressure device may be a ventilator, an anesthesia machine, an ambu bag, a continuous positive airway pressure machine, or a bi-level positive airway pressure machine.

In further embodiments, a temperature regulating device regulates the temperature of the gas flowing to the patient's lungs. In embodiments, the temperature regulating device is a cooling device, such as an ice bag, a refrigerant based device, a thermoelectric cooler, an air-to-water cooler, a carbon dioxide cooler, or any combination thereof. In other embodiments, wherein the temperature regulating device further includes a heating device. In still other embodiments, the temperature regulating device is a heating device.

In general, in another embodiment, the invention features a method for regulating a patient's body temperature including disconnecting a patient from a positive pressure device having an inspiratory limb and an expiratory limb and inserting a temperature regulating device into the inspiratory limb of the positive pressure device. The patient is reconnected to the positive pressure device and the temperature of the temperature regulating device is adjusted.

In embodiments, the method also includes monitoring the temperature of a gas flowing out of the temperature regulating device and adjusting the temperature of the gas by adjusting the temperature of the temperature regulating device.

In further embodiments, the positive pressure device may be a ventilator, an anesthesia machine, an ambu bag, a continuous positive airway pressure machine, or a bi-level positive airway pressure machine. In embodiments, the temperature regulating device is a cooling device, such as an ice bag, a refrigerant based device, a thermoelectric cooler, an air-to-water cooler, a carbon dioxide cooler, or any combination thereof. In other embodiments, the temperature regulating device further includes a heating device. In still other embodiments, the temperature regulating device is a heating device.

The other embodiments of the invention can be implemented to realize one or more of the following advantageous. The system and method provides an expedited yet substantially safe way to regulate a patient's body temperature. The system and method provide a non-invasive means to accomplish regulation of the body temperature. Cooling through the lungs provides a rapid method of cooling the core body temperature. Further, the system and method are cost-effective and practical in application, and the components can be used with a number of patients. For example, the system can be adapted to existing equipment found in most all medical facilities. Further, the system and method work easily when a patient is undergoing treatment while intubated. Also, the method enables rapid regulation in a patient's core body temperature in life-threatening or emergency situations. The system and method also reduce the time commitment and personnel training required for medical staff during treatment. The system can be used to reduce the core body temperature to treat hyperthermia or to reduce damage caused by trauma or surgical procedures. The system can also be used to elevate the core body temperature in cases of hypothermia or after reducing the core body temperature for treatment.

These and other features and advantages of the present invention will become more readily apparent from the attached drawings and the detailed description of the preferred embodiments, which follow.

### BRIEF DESCRIPTION OF THE DRAWINGS

Various embodiments of the invention will hereinafter be described in conjunction with the appended drawings provided to illustrate and not to limit the invention, where like designations denote like elements, and in which:

FIG. 1 is a perspective view of the patient cooling system shown in accordance with a preferred embodiment of the present invention;

FIG. 2 is a schematic flow diagram illustrating the interaction of different components of the patient cooling system in accordance with a preferred embodiment of the present invention;

FIG. 3 shows a typical ventilator system in use on a patient (not shown) before installation of the patient cooling system of the present invention; and

FIG. 4 is a flowchart showing an illustrative method of utilizing the patient cooling system of the present invention for monitoring and controlling the body temperature of a patient.

Like reference numerals refer to like parts throughout the several views of the drawings.

### DETAILED DESCRIPTION

Shown throughout the figures, embodiments of the present invention are generally directed towards a system and method of reducing patient body temperature in an expedited yet safe, cost-effective, and convenient manner.

An embodiment of the present invention is configured to cool core body temperature by ventilating cooled gas through the lungs of patient. More specifically, it will be appreciated by those skilled in the art relating to medical treatment that the lungs comprise a highly vascular organ with a high turnover ratio of blood per unit of time. As such, the present invention is directed towards ventilating cooled gas through the lungs of a patient in a controlled and monitored manner so as to cool the core body temperature of a patient. The lungs provide an ideal reservoir for the cooling of blood and permit a quick rate of cooling without the need for additional surgical procedures to be performed.

As the hot blood of the body is pumped through the pulmonary vascular system, it is cooled down by the cold gas being delivered into the lungs by the ventilator, or other positive pressure device such as, for example, an anesthesia machine. The cooled blood is then pumped out of the lungs and into the systemic capillary bed which is composed of the vasculature between the systemic arterial and venous systems. These capillary beds provide the vehicle for heat exchange between the blood and the tissues. The blood returns to the lungs reheated by the body's tissues and leaves the lungs re-cooled by the gas ventilating the patient. This sequence of events is repeated over and over again and, as described herein, is an effective way to reduce the temperature of the human body.

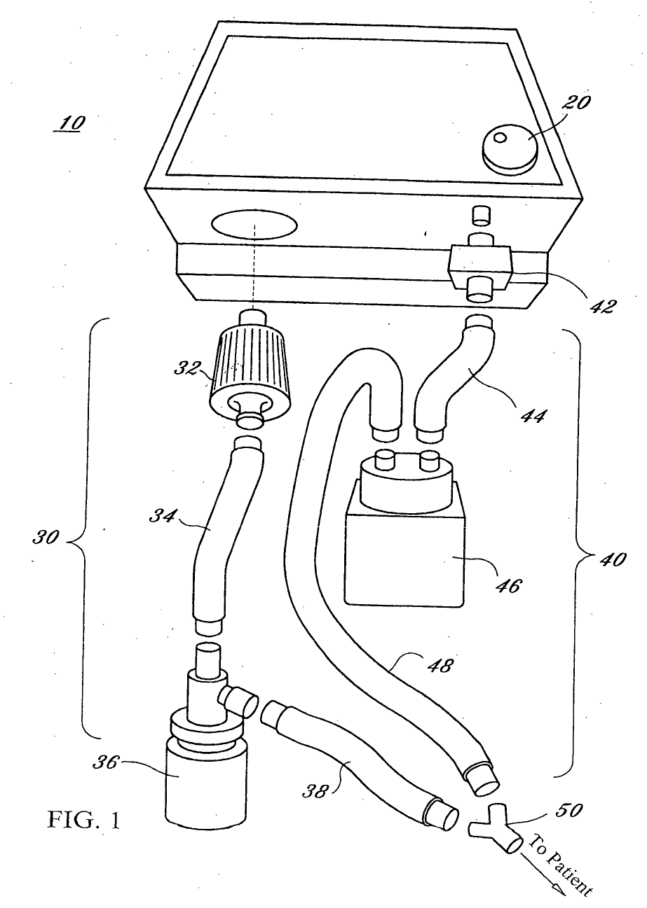

Referring to FIG. 1, a patient cooling system 10 includes a ventilator 20 to deliver a flow of gas to a patient's lungs. The ventilator 20 will preferably include a ventilator to patient line 40, known as the inspiratory limb, and a ventilator from patient line 30, known as the expiratory limb.

The expiratory limb 30 will first be described in accordance with an exemplary embodiment of the present invention. In this line 30, an expiratory filter 32 may also be provided, as shown in FIG. 1. The expiratory filter 32 is used to filter humidity, moisture and microbes from the gas as it is being expelled to the surrounding environment.

A tube 34 will extend from the expiratory filter 32 to the collector vial 36, followed by tube 38 to the patient wye 50. The collector vial 36 may be optional, and will preferably be configured to collect and remove humidity and condensation from the system.

The ventilator to patient line or inspiratory limb 40 will now be described in accordance with an exemplary embodiment of the present invention. A tube 44 extends from the inspiratory filter 42 to a cooler 46 as shown. The inspiratory filter 42 prevents contamination of components of the apparatus. The cooler 46 will be described in more detail below.

Tube 48 extends from the cooler 46 to the patient wye 50 as shown. Tube 48 comprises part of the inspiratory limb 40 of the ventilator breathing circuit. It will be appreciated by those skilled in the art to which the present invention pertains that the ventilator breathing circuit of the present invention illustrated is presented as exemplary only and that any of a wide variety of other configurations may be utilized, as desired, without departing from the scope of the present invention.

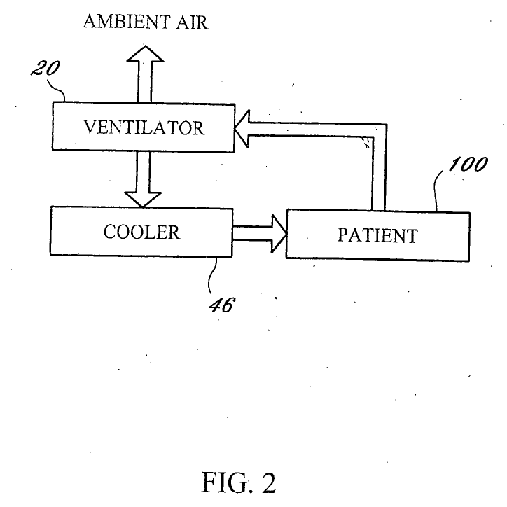

FIG. 2 is a schematic diagram illustrating the interaction of different components of the patient cooling system 10 in accordance with a preferred embodiment of the present invention. As shown, the patient cooling system 10 of the present invention cooperatively engages a ventilator 20 utilized in conjunction with a cooler 46, to cool patient 100.

The ventilator 20 of the present invention is cooperatingly engaged with a cooler 46, as shown, so that cooled gas exits the cooler 46, as desired, to the lungs of the patient 100 via the tube 48 comprising part of the inspiratory limb 40 of the ventilator breathing circuit as illustrated in FIG. 1, towards the patient via patient wye 50. As the patient 100 exhales, the return air follows the expiratory limb 30, or the ventilator from patient line, as shown. Specifically, still referring to FIG. 1, the return air exits through the tube 38 of the ventilator breathing circuit towards the collector vial 36. At this point, another tube 34 may be provided as well.

In the expiratory limb 30, an expiratory filter 32 may also be provided, as desired, as shown in FIG. 1. The tube 34 will extend from the expiratory filter 32 to the collector vial 36, followed by tube 38 comprising the expiratory limb of ventilator breathing circuit to the patient wye 50. The patient wye 50 is a component of the ventilator circuit and separates the inspiratory limb 40 from the expiratory limb 30. It will be appreciated by those skilled in the art that the purpose of the inspiratory limb 40 is to provide gas to the patient from the ventilator 20, or other positive pressure device, while the expiratory limb 30 expels gas from the patient to the outside environment.

It will be understood by those skilled in the art to which the present invention pertains that any of a wide variety of different mechanisms may be utilized for the cooler 46 of the present invention. For example, in one embodiment of the present invention, the cooler 46 may simply comprise ice or an ice bag disposed within the patient breathing circuit and configured to interact and cool gas entering a patient's lungs. The ice or ice bag may be placed in any of a wide variety of components of the patient breathing circuit such as, for example, in a humidification unit.

The cooler 46 may also comprise a thermo-electric cooler. Thermo-electric coolers are solid-state heat pumps, and have the advantage of having no moving parts and not requiring the use of harmful chlorofluorocarbons. As thermo-electric coolers have no moving parts, they are inherently reliable and require little to no maintenance. Thermo-electric coolers are space-saving and have the ability to heat as well as cool. It will be appreciated by those skilled in the art, that the cooler 46 of the present invention may be configured with the ability to heat air as well as cool air so that a patient's core body temperature can quickly and carefully be controlled.

The cooler 46 may be configured as an air to water intercooler. Air to water intercoolers provide an effective way to lower temperatures and operate similar to thermo-electric coolers.

Another preferred cooler 46 utilized with the present invention will include a carbon dioxide (CO2) cooler. Such, devices are well known and operate by spraying carbon dioxide gas onto the surface of an aluminum element. It is seen that the ventilator's 20 flow of gas is directed through the cooler 46 to reduce the temperature therein. The carbon dioxide may be sprayed onto the aluminum element in pulses while the pressure is regulated in order to effectively control the temperature of the gas. If desired, a temperature sensor may be mounted just after the cooler 46 to sample the temperature of the gas. Such readings provide data upon which decisions can be made to regulate the pulse of the carbon dioxide gas and control the regulator to keep the temperature at pre-determined levels. As the regulator controls the pressure, it is seen that by controlling the regulator, the pressure can also be controlled.

Additionally, as desired, the cooler 46 of the present invention may comprise a refrigerant-based cooling system. Such coolers are well known in the art and typically include a condenser and use a refrigerant gas such as Freon and are often utilized in existing refrigerator or air-conditioning systems.

In a most preferred embodiment, the cooler 46 may be provided with temperature indicating and control means (not shown) to indicate the temperature of gas entering and leaving the patient 100 and to provide means of adjusting the amount of cooling provided. The temperature indicating and control means may include any of a wide variety of known mechanisms such as, for example, a thermometer or number of thermometers and a standard temperature control dial that operates the cooler 46.

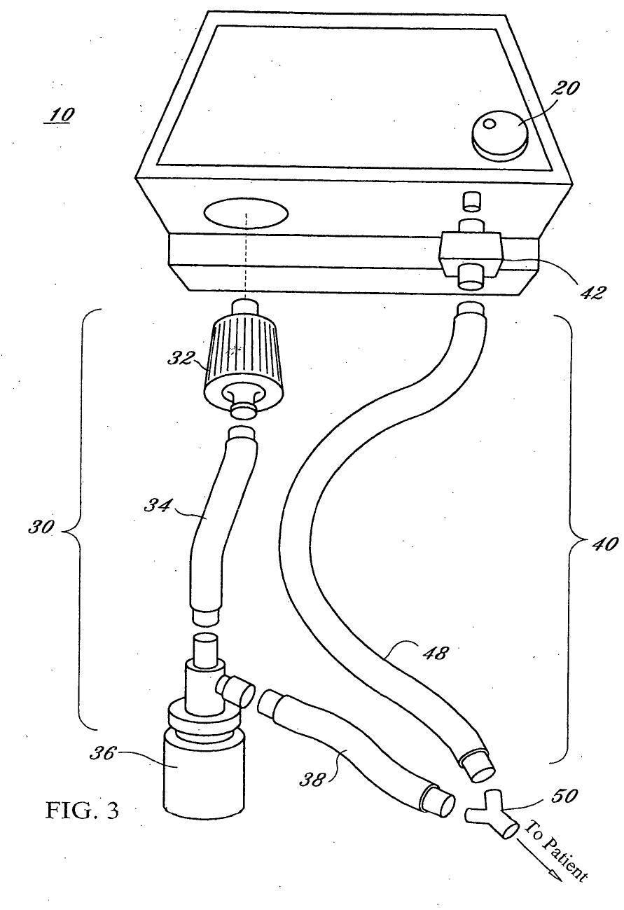

Referring to FIG. 3, a ventilator system is illustrated in use on a patient (not shown) before installation of the patient cooling system of the present invention. It will be readily understood by those skilled in the art that although a ventilator system is illustrated, any of a wide variety of other known positive pressure devices, such as an anesthesia machine, for example, may alternatively be utilized without departing from the present invention.

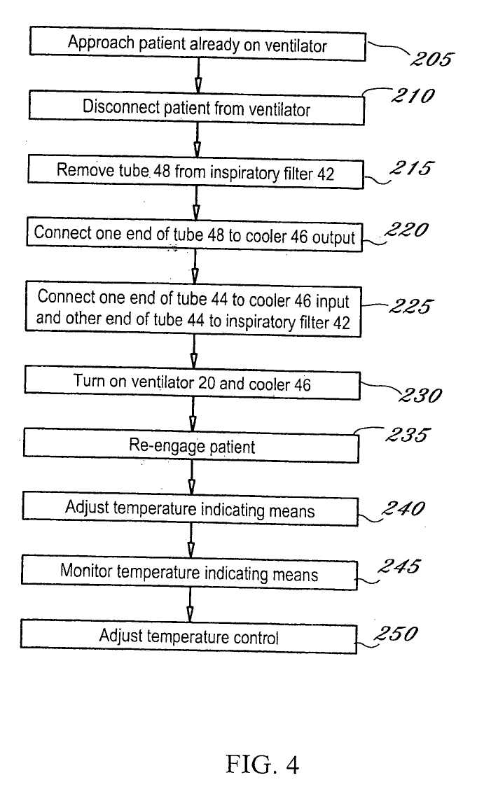

As shown in FIG. 4, the method of providing core cooling to a patient already on a ventilator will be described. As shown in FIG. 3, a typical ventilator system includes a ventilator 10 cooperatively engaging a patient via an expiratory limb 30 and an inspiratory limb 40. After approaching a patient already on a positive pressure device, such as a ventilator, as shown at step 205 of FIG. 4, the patient is disconnected from the ventilator at step 210 as will be clear to those skilled in the art to which the invention pertains. Once the patient is disconnected from the ventilator 20, it will be necessary to manually ventilate the patient as is well known in the art. Manual ventilation may be performed using an ambu bag or any of a wide variety of other known manual ventilating means or positive pressure devices.

At step 215, tube 48 is removed from the inspiratory filter 42, as shown in FIG. 3, and one end is connected to the cooler 46 output as illustrated in FIG. 1 and described at step 220 of FIG. 4. Next, one end of tube 44 is secured to the cooler 46 input and the other end of tube 44 is secured to the inspiratory filter 42 at step 225.

The ventilator 20 and cooler 46 are then turned on at step 230 and the patient is re-engaged at step 235. The temperature control means are then adjusted as desired at step 240 and the patient is monitored at step 245. Patient cooling can be adjusted periodically as desired as shown at step 250.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence. For example, while the positive pressure device has been described as a ventilator, the positive pressure device could be an anesthesia machine, an ambu bag, a continuous positive airway pressure machine, a bi-level positive airway pressure machine, or any other positive pressure device to supply gas to a patient's lungs. Further, while the positive pressure device has been described as having an inspiratory limb and an expiratory limb, the positive pressure device does not need to have either an inspiratory limb or an expiratory limb. For example, neither an ambu bag, a continuous positive airway pressure machine, nor a bi-level positive airway pressure machine has an inspiratory limb or an expiratory limb. In such embodiments, a cooler is configured to cool the gas provided by the positive pressure device chosen.

Also, while the preferred embodiment has been described as being configured to cool a patient's core body temperature, the system would work equally well to warm a patient's core body temperature. For example, the cooler may be replaced with a heater to heat the gas provided to the patient. Further, the patient cooling system may include both a cooler and a heater to allow the system to regulate a patient's core body temperature as needed.

Further, the patient is not limited to humans. The patient cooling system may be configured equally as well to be used in veterinary medicine to help various animals.

What is claimed is:

1. A temperature regulating system comprising:
a gas delivery device configured to deliver gas to a patient;
a temperature regulating device in fluid communication with the gas delivery device configured to regulate the temperature of the gas;
a temperature sensor configured to monitor the temperature of the gas; and
a temperature controller to control the temperature of the gas.

2. The temperature regulating system of claim 1 wherein the gas delivery device is a positive pressure device.

3. The temperature regulating system of claim 2 wherein the positive pressure device is selected from the group consisting of a ventilator, an anesthesia machine, an ambu bag, a continuous positive airway pressure machine and a bi-level positive airway pressure machine.

4. The temperature regulating system of claim 1 wherein the temperature regulating device is a cooling device.

5. The temperature regulating system of claim 4 wherein the cooling device is selected from the group consisting of an ice bag, a refrigerant based device, a thermo-electric cooler, an air-to-water cooler, a carbon dioxide cooler, or any combination thereof.

6. The temperature regulating system of claim 1 wherein the temperature regulating device is a heating device.

7. The temperature regulating system of claim 4 wherein the temperature regulating device further includes a heating device.

8. The temperature regulating system of claim 1 wherein the temperature sensor is selected from the group consisting of a thermometer and a thermocouple.

9. The temperature regulating system of claim 1 wherein the temperature controller is a temperature control dial connected to the temperature regulating device.

10. The temperature regulating system of claim 1 wherein the temperature controller is in communication with the temperature sensor and the temperature regulating device to regulate the temperature of the gas to a preset temperature.

11. The temperature regulating system of claim 10 further comprising a second temperature sensor configured to monitor the temperature of the gas leaving the patient.

12. The temperature regulating system of claim 1 further comprising an inspiratory limb and an expiratory limb in communication with the gas delivery device.

13. The temperature regulating system of claim 12 wherein the temperature regulating device is in fluid communication with the inspiratory limb.

14. A temperature regulating system comprising:
a positive pressure device configured to deliver gas to a patient;
an inspiratory limb and an expiratory limb in fluid communication with the positive pressure device;
a temperature regulating device in fluid communication with the inspiratory limb configured to regulate the temperature of the gas;
a temperature sensor in fluid communication with the inspiratory limb configured to monitor the temperature of the gas; and
a temperature controller in communication with the temperature regulating device to control the temperature of the gas.

15. The temperature regulating system of claim 14 where in the positive pressure device is selected from the group consisting of a ventilator and an anesthesia machine.

16. The temperature regulating system of claim 14 where in the temperature regulating device is a cooling device.

17. The temperature regulating system of claim 16 wherein the cooling device is selected from the group consisting of an ice bag, a refrigerant based device, a thermo-electric cooler, an air-to-water cooler, a carbon dioxide cooler, or any combination thereof.

18. The temperature regulating system of claim 14 wherein the temperature regulating device is a heating device.

19. The temperature regulating system of claim 16 wherein the temperature regulating device further includes a heating device.

20. The temperature regulating system of claim 14 further comprising:
an inspiratory filter in fluid communication with the inspiratory limb;
a expiratory filter in fluid communication with the expiratory limb; and
a collector vial disposed within the inspiratory limb and configured to remove humidity and condensation from the temperature regulating system.

21. The temperature regulating system of claim 14 further comprising:
an inspiratory filter in fluid communication with the inspiratory limb;
a expiratory filter in fluid communication with the expiratory limb; and
a collector vial disposed within the expiratory limb and configured to remove humidity and condensation from the temperature regulating system.

22. A method for treating a patient's body temperature comprising:
providing positive pressure to deliver a flow of gas to a patient's lungs, wherein the gas comprises a temperature; and
regulating the temperature of the gas flowing to the patient's lungs to regulate the patient's body temperature.

23. The method of claim 22 wherein providing the positive pressure comprises using a positive pressure device selected from the group consisting of a ventilator, an anesthesia machine, an ambu bag, a continuous positive airway pressure machine and a bi-level positive airway pressure machine.

24. The method of claim 22 wherein regulating the temperature of the gas comprises using a temperature regulating device.

25. The method of claim 24 wherein the temperature regulating device is a cooling device.

26. The method of claim 25 wherein the cooling device is selected from the group consisting of an ice bag, a refrigerant based device, a thermo-electric cooler, an air-to-water cooler, a carbon dioxide cooler, or any combination thereof.

27. The method of claim 24 wherein the temperature regulating device is a heating device.

28. The method of claim 25 wherein the temperature regulating device further includes a heating device.

29. A method for regulating a patient's body temperature comprising:
disconnecting a patient from a positive pressure device having an inspiratory limb and an expiratory limb;
inserting a temperature regulating device into the inspiratory limb of the positive pressure device;
reconnecting the patient to the positive pressure device; and
adjusting the temperature of the temperature regulating device.

30. The method of claim 29 further comprising:
monitoring the temperature of a gas flowing out of the temperature regulating device; and
adjusting the temperature of the gas by adjusting the temperature of the temperature regulating device.

31. The method of claim 29 wherein the positive pressure device is selected from the group consisting of a ventilator, an anesthesia machine, an ambu bag, a continuous positive airway pressure machine and a bi-level positive airway pressure machine.

32. The method of claim 29 wherein the temperature regulating device is a cooling device.

33. The method of claim 32 wherein the cooling device is selected from the group consisting of an ice bag, a refrigerant based device, a thermoelectric cooler, an air-to-water cooler, a carbon dioxide cooler, or any combination thereof.

34. The method of claim 29 wherein the temperature regulating device is a heating device.

35. The method of claim 32 wherein the temperature regulating device further includes a heating device.
